(19) United States
(12) Patent Application Publication
(10) Pub. No.: US 2025/0304728 A1
(43) Pub. Date: Oct. 2, 2025

(54) PREPARATION OF N-VINYLPYRROLIDONE POLYMERS, CONTAINING LESS THAN 0.5 WT% OF 2-PYRROLIDONE

(71) Applicant: BASF SE, Ludwigshafen (NL)

(72) Inventors: Theo Smit, Den Haag (NL); Ulrich Filges, Neustadt (DE); Monika Meller, Ludwigshafen (DE); Oliver Welz, Ludwigshafen (DE); Maike Bergeler, Ludwigshafen (NL); Felicitas Guth, Ludwigshafen (NL); Karl-Hermann Strube, Speyer (DE)

(21) Appl. No.: 18/713,726

(22) PCT Filed: Nov. 17, 2022
(86) PCT No.: PCT/EP2022/082271

§ 371 Date: May 28, 2024

(30) Foreign Application Priority Data
Nov. 30, 2021 (EP) 21211423.5

(51) Int. Cl. C08F 26/10; C08F 2/10; C08F 2/38

(52) U.S. Cl. CPC C08F 26/10; C08F 2/10; C08F 2/38

(57) ABSTRACT

Objective of the present invention to provide a process for the preparation of N-vinylpyrrolidone polymers, containing less than 0.5 wt % of 2-pyrrolidone, by solution polymerization in aqueous medium.

It is the objective of the present invention to provide a process for the preparation of N-vinylpyrrolidone polymers, containing less than 0.5 wt % of 2-pyrrolidone, by solution polymerization in aqueous medium.

DE1645642 describes the extraction of 2-pyrrolidone from aqueous polyvinylpyrrolidone solutions by halogenated solvents. Disadvantages of this method are the toxicity of the solvents and the fact that the used solvents either have to be disposed or purified by distillation.

The removal of 2-pyrrolidone from polyvinylpyrrolidone solutions by modified activated coal is claimed in CN101633706. Only coal modified with strong oxidizing agents such as hydrogen peroxide and nitric acid proved to be able to reduce the pyrrolidone content and relatively high coal loadings were required.

The preparation of N-vinylpyrrolidone polymers by free radical polymerization is known. The mechanism of polymerization under various conditions has for example been described in F. Haaf, A. Sanner, F. Straub, Polymer J. 1985, 17, 1, 143-152.

The preparation of polyvinylpyrrolidone with a low content of 2-pyrrolidone by polymerization in alcoholic solutions has been disclosed, for example, in U.S. Pat. No. 4,053,696. However, this process has the disadvantage that the used isopropanol must be either disposed or purified by distillation. The production of polyvinylpyrrolidone in organic solvents is more expensive and less environmentally sustainable than producing the polymer in a water-based process.

A widely used aqueous process for the production of polyvinylpyrrolidone involves the use of hydrogen peroxide (for example described in U.S. Pat. No. 2,335,454). In this system, hydrogen peroxide functions both as initiator and a polymerization regulator. Different molecular weights can be obtained by varying the amount of hydrogen peroxide used to prepare the polymer. However, as described in F. Haaf, A. Sanner, F. Straub, Polymer J. 1985, 17, 1, 143-152, termination by H2O2 results in the formation of a polymer-CH(pyrrolidone)-OH end group. Hydrolysis of this end group results in 2-pyrrolidone formation. Increasing the amount of hydrogen peroxide, increases the number of chain transfer events, resulting in lower molecular weight product and a larger amount of 2-pyrrolidone contaminant.

Conditions that result high polymerization termination rates such as: high polymerization temperatures, high initiator loadings and the use of chain transfer agents, tend to also result in increased impurity levels. The synthesis of polymer product with low impurity levels therefore becomes more challenging when the targeted polymer molecular weight is reduced. JP2016188268 claims a different approach for the synthesis of low molecular polyvinylpyrrolidone. In a first step, high molecular weight polyvinylpyrrolidone with a low impurity content is synthesized. In a second step, this high molecular weight product is treated with hydrogen peroxide to cleave the polymer chains. A disadvantage of this methods is the very high amount of hydrogen peroxide that is required for the second step. This makes this process unsuitable for large scale polymer production.

JP5483793, EP1219647 and CN102603949 describe the use of sulfite salts in combination with the water-soluble peroxides hydrogen peroxide and t-butyl hydroperoxide, to initiate the polymerization of N-vinylpyrrolidone in water. Radicals are formed by electron transfer from the reducing agent (sulfite) to the oxidizing agent (the peroxide). The temperature at which this occurs is lower than the temperature required for effective homolytic bond cleavage of the used peroxides. This means that these redox initiated polymerizations can be performed at relatively low temperatures which minimizes the rate of side-reactions that lead to impurity formation. The sulfite salt acts both as reducing agent for the peroxide and as chain transfer agent. The latter makes it possible to vary the polymer molecular weight by changing the amount of sulfite used in the synthesis process.

The use of the water-soluble peroxides hydrogen peroxide and t-butyl hydroperoxide may be practical as they can easily be introduced into the aqueous polymerization system. However, both these peroxides carry an OH-group. It was reasoned, that during the polymerization reaction, this OH group can be transferred to a polymer chain resulting in the formation of the described unstable polymer-CH(pyrrolidone)-OH end group which hydrolyses to afford 2-pyrrolidone. The invention described herein is the use of OH-free peroxyesters with a calculated aqueous solubility at 25° C. of less than 30 g/l and a structure shown below, in combination with a reducing agent, to produce polyvinylpyrrolidone with a very low 2-pyrrolidone content.

R1: CnH2n+1, with n=1-3, R2: CmH2m+1, with m=3-5.

The synthesis examples listed in JP5483793 all involve the use of peroxides (t-butyl hydroperoxide) with a calculated aqueous solubility at 25° C. of more than 50 g/l. This patent also only claims water-soluble hydroperoxides for the synthesis of polyvinylpyrrolidone. The initiator t-butyl peroxypivalate is mentioned in the description. However, the applicability of the combination of this initiator and a reducing agent for the synthesis of polyvinylpyrrolidone is limited because this affords pivalic acid as an undesirable by-product (C. A. Stanley, Ann. N.Y. Acad. Sci. 2004, 1033, 42-51 and E. P. Brass, Pharm. Rev. 2002, 54, 4, 589-598). The peroxyester t-butyl peroxypivalate is therefore excluded from the here described invention.

Examples of peroxyesters that can be used in the inventive process are tert-butyl peroxy-n-butyrate, tert-butyl peroxy-isobutyrate, tert-butyl peroxypropiate, tert-butyl peroxyacetate, tert-amyl peroxy-n-butyrate, tert-amyl peroxy-isobutyrate, tert-amyl peroxypropiate and tert-amyl peroxyacetate. Preferred are tert-butyl peroxy-isobutyrate, tert-butyl peroxyacetate, tert-amyl peroxy-isobutyrate and tert-amyl peroxyacetate. Most preferred are tert-butyl peroxyacetate and tert-butyl peroxy-isobutyrate. In the inventive process, between 0.0002 and 0.1 mole peroxyester are used per mole of N-vinylpyrrolidone. The preferred amount of peroxyester is between 0.0004 and 0.05 mole per mole of N-vinylpyrrolidone.

The inventive polymerizations are performed in an aqueous environment. The peroxyester with a calculated aqueous solubility at 25° C. of less than 30 g/l is added to the polymerization reactor in the form of a solution in one or more organic solvents. The polymerization medium can contain up to 20 wt % of organic solvent. The polymerization medium should only contain as much organic solvent as is necessary for dissolving and adding the peroxyester. Suitable organic solvents are solvents that are both compatible with a free-radical polymerization process and that can be removed from the aqueous polymer solution in a post-polymerization distillation process. Examples are alcohols, such as methanol, ethanol, n-propanol and isopropanol, aromatic hydrocarbons, such as toluene, xylene, cumene and ethylbenzene, ethers, such as 1,4-dioxane and tetrahydrofuran, alkanes such as hexane, heptane and isododecane and esters such as ethyl acetate and mixtures of the said solvents which are completely miscible with one another. Preferred solvents are hydrocarbons, methanol, ethanol and isopropanol and mixtures thereof. Most preferred are hydrocarbons and isopropanol and a mixture thereof. The weight ratio between the peroxyester and the total amount of organic solvent is between 1:0.2 and 1:200, preferably between 1:1 and 1:50.

In the inventive process, the peroxyester is combined with a reducing agent to generate the radicals that are needed to initiate the polymerization process. Suitable reducing agents are alkali metal and ammonium sulfites and bisulfites such as sodium sulfite, sodium bisulfite, ammonium sulfite and ammonium bisulfite, hydrated sulfite salts such as ammonium sulfite monohydrate and sodium sulfite heptahydrate, alkali metal and ammonium dithionites such as sodium dithionite, alkali metal and ammonium metabisulfites such as sodium metabisulfite and reducing sugars. A reducing sugar is a sugar that in solution contains an aldehyde or ketone group. When these sugars act as reducing agent, the aldehyde group is converted into a carboxylic acid. Examples are glucose, fructose and xylose. The reducing agent can also be prepared in situ by introducing sulfur dioxide into an aqueous solution of an alkali metal or ammonium hydroxide solution. In the inventive process, between 0.0005 and 0.2 mole reducing agent are used per mole of N-vinylpyrrolidone. The preferred amount of reducing agent is between 0.001 and 0.1 mole per mole of N-vinylpyrrolidone.

The molar ratio between peroxyester and the reducing agent is 1:0.5 to 1:20, preferably from 1:1 to 1:10.

The aqueous polymer solutions are normally prepared in such a way that they have solids content between 10 and 70% by weight. Preferred is a solids content from 15 to 60% by weight, and 20 to 55% by weight is most preferred.

The polymerization can optionally be carried out in the presence of a polymerization regulator, also referred to as chain transfer agent, to control the molecular weight of the produced polymer (The Chemistry of Radical Polymerization, 2nd Edition, 2005, Graeme Moad and David H. Solomon, Chapter 6: chain transfer, p 279-331). Suitable polymerization regulators are 2-mercaptoethanol, mercaptoacetic acid, alkali metal and ammonium sulfites, cysteine, mercaptosuccinic acid, isopropanol and alkali metal and ammonium hypophosphites. In some cases, the reducing agent that is combined with the peroxyester to initiate the radical polymerization, can also function as polymerization regulator. Examples of such compounds are ammonium and alkali metal sulfites and bisulfites. In other cases, the solvent that is used to add the peroxyester to the polymerization reactor can also function as chain transfer agent. An example is isopropanol. It is also possible to use more than one regulator.

The method to determine polymer K-values is described in “H. Fikentscher, systematics of celluloses based on their viscosity”, Cellulose-Chemie 13 (1932), 58-64 and 71-74. The K-value of polyvinylpyrrolidone produced by the inventive process is between 10 and 100 (1 weight % aqueous solution), preferably between 10 and 70 and more preferably between 15 and 50.

The polymerization is preferably carried out at a pH in the range from 6 to 10 in order to avoid hydrolysis of N-vinylpyrrolidone. It is therefore advisable to adjust the solutions of the starting materials to the suitable pH range with a suitable base, for example an aqueous ammonia solution.

The vinylpyrrolidone can be polymerized by conventional techniques, such as by batch polymerization in which polymerization components such as N-vinylpyrrolidone, peroxyester, reducing agent, optionally a polymerization regulator, optionally a base, and water are heated to the polymerization temperature. The reaction mixture is stirred at the polymerization temperature until conversion is more than 99.9%. In this process it is also possible where appropriate to add the initiator only after the polymerization temperature is reached. Further variants of the polymerization process comprise the feed methods, which are preferably used. In these variants of the process, some, or all polymerization components are added over a certain time to the polymerization reactor at the polymerization temperature. Polymerization components may also be partly included in the pre-feeding charge and partly be added overtime. Components may be added with constant or with varying feeding rates. It is also possible to add components in portions. Another method comprises using the heat generated by the polymerization reaction to reach a targeted polymerization temperature. Residual monomer can be converted by the addition of polymerization initiator at the appropriate temperature. It is also possible to hydrolyze residual monomer to 2-pyrrolidone by lowering the pH value of the aqueous solution to below 5 by the addition of an acid (as described for example described in WO 93/16114 A1). The disadvantage of the latter is that it may increase the amount of 2-pyrrolidone in the polymer product.

The polymerization reaction can be performed in an open vessel under atmospheric pressure, or in a closed vessel, where the pressure can be above or below atmospheric pressure. The temperature of the polymerization can be between 2° and 120° C., preferably between 4° and 100° C. and most preferred between 5° and 90° C.

The aqueous polyvinylpyrrolidone solutions can, where appropriate, be converted into solid powders by a prior art drying process. In those cases, it is preferred to remove the organic solvents that were used to introduce the peroxyester into the polymerization vessel, for example by steam distillation, prior to the drying process. Drying processes which are suitable for producing powdered polymers are all those suitable for drying from aqueous solution. Preferred processes are spray drying, fluidized bed drying, drum drying and belt drying, while processes which are less preferred but can also be used are freeze drying and freeze concentrating.

The N-vinylpyrrolidone polymers obtained by the inventive process have low contents of impurities such as 2-pyrrolidone and N-vinylpyrrolidone. The content of N-vinylpyrrolidone and 2-pyrrolidone contents are not more than 50 ppm and 0.5 wt % respectively, based on the amount of polymer.

The polyvinylpyrrolidone polymers prepared by the inventive process can be used in pharmaceutical, cosmetic, agricultural, food and feed, detergent, electrical and adhesive formulations.

These polymers can also be used to prepare membranes for liquid purification processes.

### Abbreviations:

tBPA=t-butyl peroxyacetate
tBPiB=t-butyl peroxyisobutyrate
tBPPv=t-butyl peroxypivalate
tBHP=t-butyl hydroperoxide
tBPEH=t-butyl peroxy-2-ethylhexanoate
NVP=N-vinyl-2-pyrrolidone
2-P=2-pyrrolidone

### EXAMPLES

### Solubility of Peresters

The solubility of liquid peroxide species in H2O at 25° C. was computed using the COSMO-RS solvation method1-2 as implemented in the COSMOtherm 2018 program.3 The program package TURBOMOLE (Version 7.5.2)4-5 was used for the required quantum-chemical calculations. The geometries of all species were computed at the TPSS level of theory6 with the triple-zeta def2-TZVP basis set7 using Grimme's D3 dispersion correction8 with Becke-Johnson damping9-10 and the COSMO solvation model11 in an ideal conductor characterized by a dielectric constant of infinity.

The 2018 BP86/def-TZVP parametrization was used in the COSMO-RS calculations based on two single-point calculations—one in an ideal conductor and one in the gas phase—at the default BP86 level of theory12-13 employing the def-TZVP basis set.14 The “iterative” option was used in the COSMO-RS solubility calculations, except for tert-butyl hydroperoxide. For the latter compound, its solubility in H2O was computed using the “slesol” option, resulting two phases. The tert-butyl hydroperoxide solubility given in Table 1 refers to the phase composition with the smaller tert-butyl hydroperoxide content. Table 1 shows the calculated solubilities of all species investigated, and Table 2 summarizes the calculated molecular geometries of the peroxide species and of the solvent H2O.

TABLE 1
Calculated solubilities in H2O at 25° C.

| | Solubility (g solute |
|---|---|
| Species | per kg solution) |
| Tert-butyl peroxyacetate | 21 |
| Tert-butyl peroxyisobutyrate | 2.1 |
| Tert-butyl peroxypivalate | 3.7 |
| Tert-butyl peroxy-2-ethylhexanoate | 0.07 |
| Tert-butyl hydroperoxide | 64 |
| H2O2 | completely miscible |

TABLE 2
Optimized geometries of the perester species and
of the solvent H2O. Coordinates are given in
Ångström (Å, 10⁻¹⁰ m).

| | |
|---|---|
| Species | Coordinates |

H2O

| | | | |
|---|---|---|---|
| O | −10.2179761 | 2.8707787 | 0.0209965 |
| H | −9.2480486 | 2.8741378 | −0.0262985 |
| H | −10.4965751 | 2.9366834 | −0.9069280 |

Tert-butyl peroxyacetate

| | | | |
|---|---|---|---|
| C | −8.0300734 | 1.0103076 | 0.3525847 |
| C | −7.4900734 | 2.2189173 | −0.3696920 |
| O | −7.7416737 | 3.3775584 | −0.1237440 |
| O | −6.6627069 | 1.7904054 | −1.3726705 |
| O | −6.1319027 | 2.9266093 | −2.1612454 |
| C | −4.6661959 | 2.9361219 | −2.0305884 |
| C | −4.2650819 | 3.1878607 | −0.5787341 |
| C | −4.3219545 | 4.1296666 | −2.9266315 |
| C | −4.0816704 | 1.6341148 | −2.5761481 |
| H | −4.7785040 | 5.0471071 | −2.5440773 |
| H | −4.6616662 | 3.9560255 | −3.9519203 |
| H | −3.2352735 | 4.2567690 | −2.9358471 |
| H | −4.3849787 | 0.7838659 | −1.9590374 |
| H | −2.9885559 | 1.6919526 | −2.5670110 |
| H | −4.4151451 | 1.4666003 | −3.6048753 |
| H | −4.7291318 | 4.1049009 | −0.2054382 |
| H | −3.1772154 | 3.2907770 | −0.5144656 |
| H | −4.5621368 | 2.3498899 | 0.0590273 |
| H | −7.6253144 | 0.0780488 | −0.0430234 |
| H | −9.1197636 | 1.0114054 | 0.2551609 |
| H | −7.7787311 | 1.1059548 | 1.4126275 |

Tert-butyl peroxyisobutyrate

| | | | |
|---|---|---|---|
| C | −7.2877864 | 0.7433287 | 0.8224196 |
| C | −8.3868717 | 0.2501640 | −0.1380378 |
| C | −6.7526874 | 2.0699393 | 0.3069233 |
| C | −7.8228459 | 0.9288326 | 2.2515089 |
| H | −8.2192895 | −0.0239705 | 2.6153669 |
| H | −8.6275535 | 1.6703038 | 2.2627843 |
| H | −7.0322868 | 1.2589169 | 2.9324900 |
| O | −7.3552945 | 3.1196748 | 0.2609697 |
| O | −5.4645958 | 1.8843028 | −0.1106730 |
| H | −7.9945421 | 0.0932901 | −1.1476342 |
| H | −9.2042680 | 0.9763055 | −0.1873921 |
| H | −8.7852263 | −0.7006275 | 0.2288140 |
| O | −4.8662002 | 3.1452649 | −0.6215212 |
| C | −4.5427727 | 2.9618387 | −2.0441140 |
| C | −3.5282804 | 1.8316008 | −2.2113369 |
| C | −3.9253222 | 4.3277193 | −2.3600241 |
| C | −5.8160485 | 2.7208747 | −2.8527224 |
| H | −3.0453948 | 4.5093027 | −1.7359644 |
| H | −4.6532594 | 5.1287314 | −2.2019380 |
| H | −3.6167612 | 4.3368539 | −3.4097263 |
| H | −6.2686349 | 1.7595284 | −2.5918316 |
| H | −5.5709265 | 2.6973993 | −3.9191688 |
| H | −6.5417263 | 3.5191610 | −2.6745149 |
| H | −2.6428114 | 2.0185066 | −1.5961375 |
| H | −3.2199752 | 1.7682974 | −3.2597901 |
| H | −3.9665338 | 0.8716379 | −1.9244894 |
| H | −6.4659024 | 0.0207325 | 0.8289705 |

Tert-butyl peroxypivalate

| | | | |
|---|---|---|---|
| C | −9.5294662 | 0.5998287 | −0.3165008 |
| C | −8.2302810 | 1.0316232 | 0.3976886 |
| H | −10.2533979 | 1.4192067 | −0.3323194 |
| H | −9.9660972 | −0.2470134 | 0.2228943 |
| H | −9.3261617 | 0.2869932 | −1.3460515 |
| C | −8.5491285 | 1.4690456 | 1.8434925 |
| C | −7.7168709 | 2.2772555 | −0.3376269 |
| C | −7.2258687 | −0.1322265 | 0.4100205 |
| H | −7.6722704 | −0.9714812 | 0.9538213 |
| H | −6.2950974 | 0.1454790 | 0.9132406 |
| H | −6.9876075 | −0.4685994 | −0.6031844 |
| O | −8.3294358 | 3.3203047 | −0.4611816 |
| O | −6.4808896 | 2.0539917 | −0.8375037 |
| H | −9.2631773 | 2.2970718 | 1.8491854 |
| H | −7.6404516 | 1.7816923 | 2.3686860 |
| H | −8.9860543 | 0.6219089 | 2.3821992 |
| O | −5.9846977 | 3.2619016 | −1.5553270 |
| C | −4.6095021 | 2.9602337 | −1.9685460 |
| C | −3.7283947 | 2.7261844 | −0.7420323 |
| C | −4.2481476 | 4.2690371 | −2.6793826 |
| C | −4.5942931 | 1.7749963 | −2.9332783 |
| H | −4.3046281 | 5.1151750 | −1.9883690 |
| H | −4.9157296 | 4.4481361 | −3.5271966 |
| H | −3.2226067 | 4.1901753 | −3.0524309 |
| H | −4.9002257 | 0.8561537 | −2.4259174 |
| H | −3.5805611 | 1.6326152 | −3.3209531 |
| H | −5.2685366 | 1.9598721 | −3.7751085 |
| H | −3.7988641 | 3.5745816 | −0.0544568 |
| H | −2.6859163 | 2.6158544 | −1.0569483 |
| H | −4.0267487 | 1.8151120 | −0.2165631 |

Tert-butyl peroxy-2-ethylhexanoate

| | | | |
|---|---|---|---|
| C | −3.7585760 | 0.0430947 | −1.0450477 |
| C | −2.3867797 | 0.5339444 | −0.5708159 |
| C | −1.3746431 | −0.6064269 | −0.4169432 |
| C | −0.0034129 | −0.1263039 | 0.0664615 |
| C | 1.0475431 | −1.2486744 | 0.1662701 |
| C | 2.4274051 | −0.7034151 | 0.5942581 |
| C | 3.5237281 | −1.7712759 | 0.6373288 |
| C | 0.5872242 | −2.2810367 | 1.1827952 |
| O | 0.1617892 | −4.5711746 | 1.5385749 |
| C | −0.9131188 | −5.3669192 | 0.9327012 |
| C | −2.1549493 | −4.5038713 | 0.7194047 |
| C | −0.4270420 | −6.0131016 | −0.3640469 |
| C | −1.1326583 | −6.4084370 | 2.0349190 |
| O | 0.3285916 | −2.0731758 | 2.3490540 |
| H | −4.4653419 | 0.8737154 | −1.1488229 |
| H | −4.1832773 | −0.6751228 | −0.3333248 |
| H | −3.6792057 | −0.4571618 | −2.0177042 |
| H | −2.4931761 | 1.0527166 | 0.3914541 |
| H | −1.9922489 | 1.2717528 | −1.2824266 |
| H | −1.2612154 | −1.1253901 | −1.3790942 |
| H | −1.7761730 | −1.3440292 | 0.2916092 |
| H | −0.1000576 | 0.3525511 | 1.0489036 |
| H | 0.3893817 | 0.6291504 | −0.6252915 |
| H | 1.1443499 | −1.7423215 | −0.8076058 |
| H | 2.3226273 | −0.2276663 | 1.5766548 |
| H | 2.6996004 | 0.0830331 | −0.1191729 |
| H | 3.2969064 | −2.5485183 | 1.3764989 |
| H | 4.4858373 | −1.3248882 | 0.9085884 |
| H | 3.6364278 | −2.2569187 | −0.3385571 |
| H | −1.9732824 | −3.7463784 | −0.0478690 |
| H | −2.4425545 | −4.0086560 | 1.6515364 |
| H | −2.9853451 | −5.1335113 | 0.3848469 |
| H | −0.2207543 | −5.2530071 | −1.1226135 |
| H | −1.2009562 | −6.6834186 | −0.7514828 |
| H | 0.4821729 | −6.5947886 | −0.1838794 |
| H | −1.4459083 | −5.9280185 | 2.9663649 |
| H | −0.2191565 | −6.9832413 | 2.2132715 |
| H | −1.9206285 | −7.0958767 | 1.7130951 |
| O | 0.5356102 | −3.4998838 | 0.5742221 |

Tert-butyl hydroperoxide

| | | | |
|---|---|---|---|
| O | 1.4294463 | −0.0837318 | 0.0221259 |
| O | −0.0467526 | −0.0530908 | 0.0436394 |
| C | −0.5133346 | 1.3393642 | 0.0779802 |
| C | 0.0051313 | 2.0320460 | 1.3372661 |
| C | −2.0297280 | 1.1362875 | 0.1294954 |
| C | −0.0872751 | 2.0676997 | −1.1961612 |
| H | −2.3129214 | 0.5659564 | 1.0195098 |
| H | −2.3814718 | 0.6088809 | −0.7625238 |
| H | −2.5190081 | 2.1140976 | 0.1722647 |
| H | 1.0016871 | 2.1636931 | −1.2425615 |
| H | −0.5171933 | 3.0743439 | −1.2091722 |
| H | −0.4384592 | 1.5276563 | −2.0816754 |
| H | −0.3048948 | 1.4811613 | 2.2310698 |
| H | −0.3989451 | 3.0480383 | 1.3948306 |
| H | 1.0965206 | 2.0947294 | 1.3172742 |
| H | 1.5947150 | −0.2227599 | −0.9303236 |

H2O2

| | | | |
|---|---|---|---|
| O | 1.3711021 | −0.0962164 | −0.5320818 |
| O | −0.0388830 | 0.0299901 | −0.1356040 |
| H | −0.3526823 | −0.8843116 | −0.2784937 |
| H | 1.3426824 | 0.2783417 | −1.4341433 |

### REFERENCES

1. Eckert, F.; Klamt, A., Fast solvent screening via quantum chemistry: COSMO-RS approach. AIChE Journal 2002, 48 (2), 369-385.
2. Klamt, A., Conductor-like Screening Model for Real Solvents: A New Approach to the Quantitative Calculation of Solvation Phenomena. The Journal of Physical Chemistry 1995, 99 (7), 2224-2235.
3. Eckert, F.; Klamt, A. COSMOtherm, Version C3.0, Release 18.01, COSMOlogic GmbH & Co. KG, Leverkusen, Germany, 2018.
4. TURBOMOLE V7.5.2, Turbomole GmbH: Karlsruhe, 2021.
5. Balasubramani, S. G.; Chen, G. P.; Coriani, S.; Diedenhofen, M.; Frank, M. S.; Franzke, Y. J.; Furche, F.; Grotjahn, R.; Harding, M. E.; Hattig, C.; Hellweg, A.; Helmich-Paris, B.; Holzer, C.; Huniar, U.; Kaupp, M.; Khah, A. M.; Khani, S. K.; Müller, T.; Mack, F.; Nguyen, B. D.; Parker, S. M.; Perlt, E.; Rappoport, D.; Reiter, K.; Roy, S.; Rückert, M.; Schmitz, G.; Sierka, M.; Tapavicza, E.; Tew, D. P.; Wüllen, C. v.; Voora, V. K.; Weigend, F.; Wodyński, A.; Yu, J. M., TURBOMOLE: Modular program suite for ab initio quantum-chemical and condensed-matter simulations. The Journal of Chemical Physics 2020, 152 (18), 184107.
6. Tao, J.; Perdew, J. P.; Staroverov, V. N.; Scuseria, G. E., Climbing the Density Functional Ladder: Nonempirical Meta—Generalized Gradient Approximation Designed for Molecules and Solids. Physical Review Letters 2003, 91 (14), 146401.
7. Weigend, F.; Ahlrichs, R., Balanced basis sets of split valence, triple zeta valence and quadruple zeta valence quality for H to Rn: Design and assessment of accuracy. Physical Chemistry Chemical Physics 2005, 7 (18), 3297-3305.
8. Grimme, S.; Antony, J.; Ehrlich, S.; Krieg, H., A consistent and accurate ab initio parametrization of density functional dispersion correction (DFT-D) for the 94 elements H—Pu. The Journal of Chemical Physics 2010, 132 (15), 154104.
9. Johnson, E. R.; Becke, A. D., A post-Hartree-Fock model of intermolecular interactions. The Journal of Chemical Physics 2005, 123 (2), 024101.
10. Becke, A. D.; Johnson, E. R., A density-functional model of the dispersion interaction. The Journal of Chemical Physics 2005, 123 (15), 154101.
11. Klamt, A.; Schüürmann, G., COSMO: a new approach to dielectric screening in solvents with explicit expressions for the screening energy and its gradient. Journal of the Chemical Society, Perkin Transactions 2 1993, (5), 799-805.
12. Perdew, J. P., Density-functional approximation for the correlation energy of the inhomogeneous electron gas. Physical Review B 1986, 33 (12), 8822-8824.
13. Becke, A. D., Density-functional exchange-energy approximation with correct asymptotic behavior. Physical Review A 1988, 38 (6), 3098-3100.
14. Schäfer, A.; Huber, C.; Ahlrichs, R., Fully optimized contracted Gaussian basis sets of triple zeta valence quality for atoms Li to Kr. The Journal of Chemical Physics 1994, 100 (8), 5829-5835.

### Analytical Methods:

The K values were determined at 25° C. using a 1 weight % aqueous solution. The method is described in “H. Fikentscher, systematics of celluloses based on their viscosity”, Cellulose-Chemie 13 (1932), 58-64 and 71-74. Concentrations of NVP and 2-P were determined by the Liquid Chromatography methods described in European Pharmacopoeia 10.0 under Povidone. The turbidity of polymer solutions was determined using a Hach TL2360 Turbidimeter at 23° C. Pivalic acid concentrations were determined by high pressure liquid chromatography at 40° C. using 0.5 mM H2SO4 as eluent and a conductivity detector.

### Preparation of Inventive Polymer P1:

A two-liter glass reactor, equipped with a mechanical stirrer, a condenser, a nitrogen sweep, a thermometer and inlets for the gradual additions of monomer and initiator, was charged with 450.0 grams demineralized water and 2.0 grams of a 25% aqueous ammonia solution. The following solutions were prepared: i) a monomer feed, consisting of 500.0 grams of N-vinylpyrrolidone and 170.0 grams of demineralized water, ii) a peroxide feed, consisting of 10.0 grams of a 50% solution of t-butyl peroxyacetate in hydrocarbons and 50.0 grams of isopropanol and iii) a reducing agent feed, consisting of 14.7 grams of a 34% aqueous ammonium sulfite solution, 50.0 grams of demineralized water and 0.7 gram of an 25% aqueous ammonia solution. The reactor charge was stirred at 120 rpm and heated to 70° C. under a nitrogen sweep. When 70° C. was reached, the monomer solution was added in 3 hours and the peroxide and reducing agent feeds were added in 3.5 hours. All feeds were added at constant feeding rate. After completion of the peroxide and the reducing agent feeds, the reactor temperature was increased to 85° C. and was stirred at this temperature for 2 hours. Steam was subsequently led into 1000 grams of the obtained polymer solution and condensed volatiles were collected in a separate flask. When 275 ml of distillate was collected, 0.75 grams of formic acid were added to the polymer solution. The steam distillation was discontinued when 350 ml of distillate had been collected.

Polymer P2 was prepared by using the same polymerization procedure as described for P1 with the exception that 12.1 grams of a 50% solution of t-butyl peroxy-isobutyrate in hydrocarbons instead of 10.0 g of a 50% solution of t-butyl peroxyacetate was used in the peroxide feed.

Polymer P3 was prepared by using the same polymerization procedure as described for P1 with the exceptions that no isopropanol was included in the peroxide feed and that the 10.0 grams of t-butyl peroxyacetate hydrocarbon solution was added in 1-gram portions, every 21 minutes. The first gram was added 21 minutes after the start of the monomer and reducing agent feeds and the final gram was added 189 minutes after the start of the monomer and reducing agent feeds.

### Preparation of Comparative Polymers C1-C3:

Polymer C1 was prepared by using the same polymerization procedure as described for P1 with the exception that 8.8 grams of tBPPv (75% in hydrocarbons) were used instead of the 10.0 grams of tBPA solution.

Polymer C2 was prepared by using the same polymerization procedure as described for P1 with the exception that the peroxide feed consisted of 4.9 grams of tBHP (70% in water) dissolved in 50.0 grams of water instead of 10.0 grams of tBPA solution, dissolved in 50.0 g of isopropanol.

Polymer C3 was prepared by using the same polymerization procedure as described for P1 with the exception that 8.4 grams of tBPEH (98%) was used instead of the 10 grams of tBPA solution.

TABLE 3
Comparison of different peroxides.

| | | | | | 2-P formed | | |
|---|---|---|---|---|---|---|---|
| | | | | 2-P | during | Pivalic | |
| | | | S.C. | (wt | polymerization | acid | |
| Example | Peroxide^{a} | K-value | (%)^{b} | %)^{c} | (%)^{d} | (ppm)^{c} | NTU^{e} |
| P1 | tBPA | 32 | 36 | 0.15 | 0.19 | — | 1 |
| P2 | tBPIB | 30 | 30 | 0.11 | 0.14 | — | 1 |
| P3 | tBPA | 34 | 39 | 0.16 | 0.18 | — | 1 |
| C1 | tBPPV | 28 | 36 | 0.13 | 0.13 | 200 | 1 |
| C2 | tBHP | 37 | 36 | 0.21 | 0.35 | — | 1 |
| C3 | tBPEH | 28 | 31 | 0.13 | 0.19 | — | 33 |

Residual NVP in polymer solution < 10 ppm in all cases.
^{a}The same molar amount of peroxide was used
in all experiments (7.6 mmol peroxide on 100 g NVP).
^{b}Solids content.
^{c}Concentration in solution.
^{d}The used NVP contained 0.23 wt % 2-P. This amount was subtracted from the total amount of 2-P to obtain the “formed during polymerization” value using the formula: ((2-P solution/S.C.) × 100) − 2-P from NVP = 2-P formed during polymerization. This value is on solids.
^{e}Nephelometric Turbidity Units (NTU) of polymer solution at the given concentration (s.c.).

Comparative Polymer C1 has as disadvantage, that the use of tBPPv in combination with a reducing agent leads to the formation of significant amounts of pivalic acid/ammonium pivalate (560 ppm on solids). Comparative Polymer C2 has as disadvantage, that the use of OH-functionalized tBHP results in the formation of higher amounts of 2-P in comparison to OH-free peroxides. The disadvantage in the case of C3 is that the large hydrophobic group of tBPEH peroxide leads to the formation of a water-insoluble product fraction which causes turbidity. Inventive polymers P1, P2 and P3 contain considerably lower amounts of impurities and afford clear aqueous solutions.

TABLE 4
Variation of initiator system amounts to obtain different polymer K values.

| | Peroxide | Ammonium | | | 2-P in | | 2-P |
|---|---|---|---|---|---|---|---|
| | (mmol on | sulfite (mmol | | | used | | formed |
| | 100 g | on 100 g | S.C. | K- | NVP | 2-P | during |
| Example | NVP) | NVP) | (%)^{a} | value | (%) | (%)^{b} | poly. (%)^{c} |
| C4 | tBHP (15.1) | 17.2 | 36 | 28 | 0.18 | 0.24 | 0.49 |
| P4 | tBPA (15.1) | 17.2 | 36 | 25 | 0.18 | 0.16 | 0.26 |
| P5 | tBPA (3.8) | 12.9 | 37 | 26 | 0.23 | 0.12 | 0.09 |
| P6 | tBPA (3.8) | 4.3 | 35 | 39 | 0.23 | 0.12 | 0.11 |
| P7 | tBPA (3.8) | 21.5 | 37 | 21 | 0.23 | 0.13 | 0.12 |
| P8 | tBPA (3.8) | 30.1 | 37 | 18 | 0.23 | 0.13 | 0.13 |

Residual NVP in polymer solution < 10 ppm in all cases.
^{a}Solids content.
^{b}Concentration in solution.
^{c}The amount of 2-P in the used NVP was subtracted from the total amount of 2-P to obtain the “formed during polymerization” value using the formula: ((2-P solution/S.C.) × 100) − 2-P from NVP = 2-P formed during polymerization. This value is on solids.

Polymer C4 was prepared by using the same polymerization procedure as described for C2 with the exception that the reactor was charged with 400.0 instead of 450.0 grams of demineralized water and double amounts of all raw materials were used, both for the tBHP and the ammonium sulfite feed.

Polymer P4 was prepared by using the same polymerization procedure as described for C4 with the exception that the peroxide feed consisted of 20.0 grams of a 50% solution of t-butyl peroxyacetate in hydrocarbons and 100.0 grams of isopropanol instead of 9.7 g of tBHP (70% in water) dissolved in 100.0 g of water.

Polymer P5 was prepared by using the same polymerization procedure as described for P1 with the exception that 490.0 instead of 450.0 grams of demineralized water and no aqueous ammonia solution were included in the pre-feeding reactor charge, the peroxide feed consisted of 5.0 grams of a 50% solution of t-butyl peroxyacetate in hydrocarbons and 25.0 grams of isopropanol instead of 10.0 grams of a 50% solution of t-butyl peroxyacetate in hydrocarbons and 50.0 grams of isopropanol and the reducing agent feed consisted of 22.1 grams of a 34% aqueous ammonium sulfite solution, 50.0 grams of demineralized water and 1.0 gram of an 25% aqueous ammonia solution instead of 14.7 grams of a 34% aqueous ammonium sulfite solution, 50 grams of demineralized water and 0.7 gram of an 25% aqueous ammonia solution.

Polymer P6 was prepared by using the same polymerization procedure as described for P5 with the exception that the reducing agent feed consisted of 7.4 grams of a 34% aqueous ammonium sulfite solution, 50.0 grams of demineralized water and 0.3 gram of an 25% aqueous ammonia solution instead of 22.1 grams of a 34% aqueous ammonium sulfite solution, 50.0 grams of demineralized water and 1.0 gram of an 25% aqueous ammonia solution.

Polymer P7 was prepared by using the same polymerization procedure as described for P5 with the exception that the reducing agent feed consisted of 36.8 grams of a 34% aqueous ammonium sulfite solution, 50.0 grams of demineralized water and 1.3 grams of an 25% aqueous ammonia solution instead of 22.1 grams of a 34% aqueous ammonium sulfite solution, 50.0 grams of demineralized water and 1.0 gram of an 25% aqueous ammonia solution.

Polymer P8 was prepared by using the same polymerization procedure as described for P5 with the exception that the reducing agent feed consisted of 51.5 grams of a 34% aqueous ammonium sulfite solution, 50.0 grams of demineralized water and 1.8 grams of an 25% aqueous ammonia solution instead of 22.1 grams of a 34% aqueous ammonium sulfite solution, 50.0 grams of demineralized water and 1.0 gram of a 25% aqueous ammonia solution.

Polymer P9 was prepared using the same polymerization procedure as described for P4 with the exceptions that 10 instead of 20 grams of 50% solution of t-butyl peroxyacetate in hydrocarbons was used in the peroxide feed, and 32.9 instead of 29.4 grams of 34% aqueous ammonium sulfite solution and 1.4 instead of 1.2 grams of 25% aqueous ammonia solution were used in the reducing agent feed.

Polymer P10 was prepared using: 800.0 grams demineralized water, 400.0 grams of NVP and 1.6 grams of ammonia in the pre-feeding charge, 8.0 grams of 50% solution of t-butyl peroxyacetate in hydrocarbons and 100.0 grams of isopropanol as peroxide feed and a reducing agent feed consisting of 26.3 grams 34% aqueous ammonium sulfite solution, 1.2 grams 25% aqueous ammonia solution and 19.4 grams demineralized water. The polymerization was performed at 60 instead of 70° C. The peroxide and reducing agent feeds were added in 2 hours.

Polymer P11 was prepared using: 400.0 grams demineralized water and 2.0 grams of ammonia in the pre-feeding charge, a monomer feed consisting of 500.0 grams of NVP and 170 grams of demineralized water, 18.4 grams of 50% solution of t-butyl peroxyacetate in hydrocarbons and 100.0 grams of isopropanol as peroxide feed and a reducing agent feed consisting of 34.6 grams 34% aqueous ammonium sulfite solution, 1.4 grams 25% aqueous ammonia solution and 100.0 grams demineralized water.

Polymer P12 was prepared using: 800.0 grams demineralized water, 400.0 grams of NVP and 1.6 grams of ammonia in the pre-feeding charge, 14.7 grams of 50% solution of t-butyl peroxyacetate in hydrocarbons and 100.0 grams of isopropanol as peroxide feed and a reducing agent feed consisting of 27.7 grams 34% aqueous ammonium sulfite solution, 1.2 grams 25% aqueous ammonia solution and 19.4 grams demineralized water. The polymerization was performed at 60 instead of 70° C. The peroxide and reducing agent feeds were added in 2 hours.

TABLE 5
Monomer in pre-feeding charge in comparison to monomer fed over time.

| | tBPA | Ammonium | | | | 2-P in | | 2-P |
|---|---|---|---|---|---|---|---|---|
| | (mmol on | sulfite (mmol | | | | used | | formed |
| | 100 g | on 100 g | | S.C. | K- | NVP | 2-P | during |
| Example | NVP) | NVP) | NVP | (%)^{a} | value | (%) | (%)^{b} | poly. (%)^{c} |
| P9 | 7.6 | 19.8 | feed | 28 | 22 | 0.23 | 0.09 | 0.09 |
| P10 | 7.6 | 19.8 | pre- | 27 | 30 | 0.25 | 0.09 | 0.08 |
| | | | feeding | | | | | |
| | | | charge | | | | | |
| P11 | 13.9 | 20.8 | feed | 42 | 21 | 0.21 | 0.17 | 0.19 |
| P12 | 13.9 | 20.8 | pre- | 27 | 36 | 0.16 | 0.06 | 0.06 |
| | | | feeding | | | | | |
| | | | charge | | | | | |

Residual NVP in polymer solution < 10 ppm in all cases.
^{a}Solids content.
^{b}Concentration in solution.
^{c}The amount of 2-P in the used NVP was subtracted from the total amount of 2-P to obtain the “formed during polymerization” value using the formula: ((2-P solution/S.C.) × 100) − 2-P from NVP = 2-P formed during polymerization. This value is on solids.

1. A free-radical polymerization process comprising polymerizing N-vinylpyrrolidone in an aqueous system initiated by an initiating system consisting of:
a. one or more peroxyester A, with a calculated aqueous solubility at 25° C. of less than 30 g/l, represented by general formula (1), with R1=CnH2n+1, with n=1-3 and R2=CmH2m+1, with m=3-5,
b. one or more organic solvent B, used for dissolution of the peroxyester A and
c. one or more reducing agent C.

2. The process according to claim 1 wherein a molar ratio between the peroxyester A and the reducing agent C is 1:0.5 to 1:20.

3. The process according to claim 1 wherein a weight ratio between the peroxyester A and the organic solvent B is between 1:0.2 and 1:200.

4. The process according to claim 1 wherein the organic solvent B is a hydrocarbon or a mixture of hydrocarbons.

5. The process according to claim 1 wherein the organic solvent B is a mixture of one or more hydrocarbons and alcohols.

6. The process according to claim 5 wherein the organic solvent B is a mixture of hydrocarbons and isopropanol.

7. The process according to claim 1 wherein a weight percentage of organic solvent B in the sum of organic solvent B and water used in the polymerization process is less than 20.

8. The process according to claim 1 wherein peroxyester A is t-butyl peroxyacetate.

9. The process according to claim 1 wherein the peroxyester A is t-butyl peroxy-isobutyrate.

10. The process according to claim 1 wherein the reducing agent C is ammonium sulfite.

11. The process according to claim 1 wherein the reducing agent C is a reducing sugar.

12. The process according to claim 1 wherein the reducing agent C is added as an aqueous solution.

13. The process according to claim 1, wherein additionally a polymerization regulator is added.

14. The process according to claim 1 wherein a K value of the produced polyvinylpyrrolidone is between 10 and 70.

15. The process according to claim 1 wherein a K value of the produced polyvinylpyrrolidone is between 15 and 50.

16. A polyvinylpyrrolidone obtained by a process according to claim 1 has having a residual monomer content of vinylpyrrolidone of not more than 50 ppm and a 2-pyrrolidone content of not more than 0.5 weight percent.

17. Cosmetic or pharmaceutical preparations, preparations of agroactives, preparations in the sector of food, feed, food supplementation or feed supplementation, preparations of membranes for liquid purification, adhesive preparations comprising a polyvinylpyrrolidone according to claim 16.

18. A membrane for purification of liquids comprising a vinylpyrrolidone polymer according to claim 16.
